(19) United States
(12) Patent Application Publication
(10) Pub. No.: US 2004/0112376 A1
(43) Pub. Date: Jun. 17, 2004

(54) Method of inducing euglycemia in diabetic patients

(75) Inventors: Rochelle L. Chaiken, New Rochelle, NY (US); Thomas R. Strack, New York, NY (US)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 10/727,841

(22) Filed: Dec. 4, 2003

Related U.S. Application Data
(60) Provisional application No. 60/433,221, filed on Dec. 13, 2002.

(51) Int. Cl. A61M 16/00

(52) U.S. Cl. 128/200.24; 128/203.12

(57) ABSTRACT

Inhaled insulin, relative to subcutaneously and/or transdermally administered insulin, can be used to reduce hepatic glucose output in fasted diabetic patients.

This application claims the benefit of U.S. Patent Application No. 60/433,221, filed Dec. 13, 2002.

### FIELD OF THE INVENTION

This invention is directed to a method of reducing hepatic glucose output in a diabetic patient who is using exogenous insulin to control blood sugars, and who is taking said insulin by other than a pulmonary route of administration, comprising administering said insulin to said patient by the pulmonary route, i.e. as inhaled insulin. Additionally, the invention relates to switching a patient to inhaled insulin who is at risk for developing high variability in blood sugar concentration, such that the patient is at risk of being outside euglycemia.

### BACKGROUND OF THE INVENTION

Diabetes mellitus is a serious metabolic disease that is defined by the presence of chronically elevated levels of blood glucose. Classic symptoms of diabetes mellitus in adults are polyuria and polydipsia together with elevated levels of plasma glucose. Normal fasting plasma glucose concentrations are less than 110 milligrams per deciliter. In diabetic patients, fasting concentrations are found to be at or above 126 milligrams per deciliter. In general, diabetes mellitus develops in response to damage to, or to defects in, the beta cells of the pancreas. The pancreatic beta cells deliver almost all insulin to the portal circulation and, hence, directly to the liver. The liver normally removes 50-80% of the insulin before insulin can reach other organs thus ensuring relatively low peripheral insulin levels, and appropriate insulinization of the liver to regulate hepatic glucose output.

Primary diabetes mellitus is classified as Type 1 diabetes (also called insulin-dependent diabetes mellitus or IDDM) and Type 2 diabetes mellitus (also called non-insulin dependent diabetes mellitus or NIDDM). Type I (juvenile onset or insulin-dependent) diabetes is a well-known hormone deficient state, in which the pancreatic beta cells appear to have been destroyed by the body's own immune defense mechanisms. Patients with Type I diabetes mellitus have little or no endogenous insulin secretory capacity. These patients develop extreme hyperglycemia. Type I diabetes was fatal until the introduction of insulin replacement therapy some 70 years ago—first using insulins from animal sources, and more recently, using human insulin made by recombinant. DNA technology. Typically, the liver is exposed to very low levels of insulin in Type 1 diabetes while peripheral insulin levels tend to be much higher than in healthy subjects.

Type 2 diabetes is characterized by insulin resistance, i.e., a failure of the normal metabolic response of peripheral tissues to the action of insulin, compounded by a relative insulin deficiency. In other words, insulin resistance is a condition where the circulating insulin produces a subnormal biological response. In clinical terms, insulin resistance is present when normal or elevated blood glucose levels persist in the face of normal or elevated levels of insulin. The hyperglycemia associated with Type 2 diabetes can sometimes be reversed or ameliorated by diet or weight loss sufficient to restore the sensitivity of the peripheral tissues to insulin. Progression of Type 2 diabetes mellitus is associated with increasing concentrations of blood glucose and coupled with a relative decrease in the rate of glucose-induced insulin secretion. Thus, for example, in late-stage Type 2 diabetes mellitus, there may be an insulin deficiency. Typically, the liver in Type 2 diabetes is exposed to normal insulin levels but does not respond appropriately because of resistance to insulin.

The timewise progressions of Type I versus Type 2 diabetes can (and usually do) differ markedly. Youthful (e.g., pediatric) patients suffering from Type I diabetes may not have their condition diagnosed until after the bulk of pancreatic beta cells have been destroyed, thereby necessitating chronic insulin therapy. Usually, Type I progresses on the order of a couple of years before the pancreas has been damaged to the point that it no longer produces sufficient insulin to meet the patient's metabolic needs. In contrast to Type I diabetes, treatment of Type 2 diabetes frequently does not require the use of insulin, and the condition itself can progress over several decades. Institution of therapy in Type 2 diabetes usually involves a trial of dietary therapy and lifestyle modification, typically for 6-12 weeks in the first instance. Features of a diabetic diet include an adequate but not excessive total calorie intake, with regular meals, restriction of the content of saturated fat, a concomitant increase in the polyunsaturated fatty acid content, and an increased intake of dietary fiber. Lifestyle modifications include the maintenance of regular exercise, as an aid both to weight control and also to reduce the degree of insulin resistance. As mentioned above, in late-stage Type 2 diabetes mellitus, there may be a frank insulin deficiency such that, ultimately, a Type 2 patient will require the administration of exogenous insulin to aid in controlling his or her sugar metabolism.

Whether classified as Type 1 or Type 2, diabetic patients suffer special medical problems not characteristic of most non-diabetics. Among the most severe are (1) chronically high levels of fasting glucose and (2) wide ranges of fasted blood glucose concentrations. This is particularly true of diabetic patients who self administer insulin by the subcutaneous route, the most common route of administration used by diabetics, although these conditions could also be developed by diabetics who take insulin by a transdermal route. Thus it is important to maintain blood sugars within a certain, relatively narrow range when a diabetic patient is in a fasted state, particularly in the period of 6 to 8 hours since food was last eaten, and after. A problem which this invention addresses is that of maintaining blood sugars below a certain level when the patient is fasted, as discussed in greater detail below.

### SUMMARY OF THE INVENTION

This invention provides a method of reducing hepatic glucose production more potently and for a longer duration, thereby inducing euglycemia in a diabetic patient who is switched to using insulin delivered by the pulmonary route, i.e., as inhaled insulin, to control blood sugars, from using insulin by a subcutaneous and/or transdermal route of administration.

Thus, in a first aspect, the invention provides a method of inducing euglycemia by reducing hepatic glucose production in a diabetic patient in need of such treatment who is using exogenous insulin to control blood sugars and who is taking said insulin by 1 or more daily subcutaneous and/or transdermal administrations, comprising administering said insulin to said patient by the pulmonary route.

Insulin delivered by a pulmonary route is sometimes referred to herein (including the claims) as “pulmonary insulin”, which is a synonym for “inhaled insulin”.

The phrase “euglycemia” as employed herein has its conventional medical meaning, i.e., a normal blood glucose concentration, a synonym being normoglycemia (Stedman's Concise Medical Dictionary For The Health Professions, Third Edition, John H. Dirckx, M. D., Editor, Published by Williams & Wilkins). In general, it is desired to maintain a euglcemic (i.e., “normal”) blood glucose level between 70 and 140 mg/dL when fasting. An accepted clinical alternative to determining blood glucose is the measurement of the glycosylated hemoglobin fraction (HbA1 c) which represents a long-term (4-6 week) “memory” of increased blood glucose as glucose irreversibly binds to hemoglobin and in increasing amounts with rising blood glucose. The “normal” range of HbA1c is 4-6.5%. The advantage of HbA1 c lies in the fact that, unlike blood glucose, it is more than a point estimate but well correlates with exposure to hyperglycemia over an extended period of time (4-6 weeks) and the incidence of late complications of diabetes such as eye disease.

The phrase “comprising administering said insulin to said patient by the pulmonary route” means changing, partly or wholly, the route of administration in a patient who has been taking exogenous insulin via the transdermal or subcutaneous route to the pulmonary route. Thus a patient who self-administers insulin subcutaneously or transdermally several times a day would have his or her regimen changed to one in which 1 or more (including all) of those daily administrations is switched to pulmonary administration. The guideline for changing over to pulmonary administration is when the proportion of glycosylated hemoglobin (HbA1 c) exceeds 7% (the percentage being based on the total amount of hemoglobin in the blood), although the physician can use his professional discretion as well to modify a regimen for a given patient. As just noted, 7% HbA1 c represents a threshold level or trigger point for initiating or for instituting new therapy based on 2 landmark studies (DCCT, UKPDS). See American Diabetes Association: Standards of Medical Care for Patients With Diabetes Mellitus. Diabetes Care 25:S33-S49, 2002; New England Journal of Medicine 1993; 329: 977-986; the British Medical Journal 2000; 321: 405-412; The Lancet 1998; 352: 837-853; and The Lancet 1998; 352: 854-865. In diabetic patients, insulin is typically administered transdermally or subcutaneously (SC) several times a day, an optimal regimen being a SC administration before each meal and, optionally, a SC administration prior to bedtime. As noted, a physician can implement or modify the regimen depending on the needs of the individual patient. Once a patient shows a need to block sugar production in view of excess glucose production beyond 7% HbA1c, an election can be made to switch one or more daily SC and/or transdermal administrations to inhaled insulin, thereby correspondingly reducing hepatic glucose output. Thus a patient who is administering insulin solely by subcutaneous injection, for example, may, within the scope of the invention, be switched to a regimen which entails taking insulin by, for instance, a single injection of subcutaneous long-acting insulin plus pre-meal inhaled insulin.

Thus the invention is focused, inter alia, on reducing hepatic glucose production during the period when a patient is fasted, including when the patient is asleep. A proportion of such patients experience being outside of euglycemia during such fasted periods, i.e., they are, to varying degrees depending on the patient, outside the “normal” range of 70 to 140 mg/dL and/or in excess of the upper limit of normal for HbA1 c (depending on laboratory standards, usually <7%). In such patients the major source of glucose production is the liver. Reduction or elimination of hepatic glucose production is believed to remove a source of glucose production which, in some patients and if left unchecked, would otherwise push blood glucose levels outside the aforementioned range of 70 to 140 mg/dL and/or above 7% HbA1c. Such unchecked (hepatic) glucose production is believed to be in large part responsible for patients being outside euglycemia. By switching one or more daily transdermal and/or subcutaneous insulin administrations in such patients over to inhaled insulin administration, hepatic glucose production and, correspondingly, variability in blood glucose levels above normal levels, can be reduced. Thus, a patient who is susceptible to being outside euglycemia and who is taking insulin subcutaneously, is likely to see a his or her blood glucose levels normalize by switching one or more of his or her daily subcutaneous and/or transdermal insulin administration(s) over to inhaled insulin as the form of administration.

The medical disadvantages of being outside euglycemia (i.e., above the value of 7% HbA1c and/or above a postprandial blood glucose concentration of 140 mg/dL) can be considerable. The greater the extent to which a patient is outside euglycemia, the more the patient risks effecting abnormal metabolic pathways and generating abnormal metabolites. Such pathways can contribute to the deterioration of nervous and vascular tissue, which is clinically manifested as neuropathy (degeneration of the peripheral nervous system), ultimately leading to muscle weakness, neuropathic pain, atrophy, and possibly contributing to the amputation of lower limbs.

The invention thus provides a method for reducing hepatic glucose output in a diabetic patient who has been taking exogenous insulin either subcutaneously and/or transdermally and who is, who has been, or who is at risk of being, outside euglycemia. In different words, the invention is directed to maintaining a more physiological range of blood glucose concentrations, below 140 mg/dL and/or an HbA1c below 7%, by using inhaled insulin relative to using insulin that has been administered transdermally or subcutaneously in patients who are frequently or chronically below the aforementioned numbers. As mentioned, and while not wishing to be bound by theory, it is believed that switching (or initiating) a patient to (or on) inhaled insulin aids in helping the body to block hepatic glucose output, thereby limiting larger elevations in blood glucose concentration effected with transdermal or subcutaneous administration.

In a principle embodiment, the patient taking insulin by the transdermal or subcutaneous route is simply switched to a new regimen, which requires, in whole or in part, the patient to take his or her insulin via the pulmonary route. The invention is thus considered to be especially applicable to switching diabetic patients, who are already wholly on a regimen of either subcutaneous and/or transdermal insulin, and who are actually outside euglycemia, wholly or partially over to pulmonary insulin. It is particularly preferred to practice the invention by switching patients who suffer from being outside euglycemia from a regimen of subcutaneously administered insulin over to a regimen which requires, in whole or in part, that one or more subcutaneous or transdermal daily doses of insulin be administered as inhaled insulin.

A “patient who is taking insulin” or who is “using exogenous insulin to control blood sugars” refers to patients who have are already on a regimen of subcutaneous or transdermally administered insulin. A patient who is “at risk of being outside euglycemia” refers, for example, to one who has been diagnosed previously as being outside euglycemia, as well as to those who are presently known to suffer from being outside euglycemia after having already started on a regimen of (subcutaneous or transdermal) insulin to aid in controlling blood sugars. The invention is accordingly applicable to Type 2 as well as Type 1 diabetics.

“Insulin” means the art-recognized polypeptide used in the treatment of diabetics in a substantially purified form, and also the various commercially available forms, which include excipients. The term encompasses natural extracted human insulin, recombinantly produced human insulin, insulin extracted from bovine and/or porcine sources, recombinantly produced porcine and bovine insulin, and mixtures thereof. The term “insulin” is also intended to encompass insulin analogs wherein one or more of the amino acids within the polypeptide chain has been replaced with an alternative amino acid and/or wherein one or more of the amino acids has been deleted or wherein one or more additional amino acids has been added to the polypeptide chain. In general, such insulin analogs of the present invention include “super insulin analogs” wherein the ability of the insulin analog to affect serum glucose levels is substantially enhanced as compared with conventional insulin as well as hepatoselective insulin analogs, which are more active in the liver than in adipose tissue. The invention may employ an aerosolized inhaled insulin, which is monomeric, such as insulin lispro.

Administering insulin by the “pulmonary route” or as “pulmonary insulin” means administering insulin as inhaled insulin. “Inhaled insulin”, in turn, means an aerosolized, wet or dry, particulate or droplet, insulin-containing formulation which is administered by having the patient “breathe” an insulin-containing aerosol into the lungs, generally by drawing the aerosol through the mouth and into the deep lung. The formulation can, for example, comprise dry particles inhaled from a dry powdered inhaler such as that available from Inhale Therapeutics Systems, San Carlos, Calif. The inhaled insulin formulation can also be a particulate, insulin-containing formulation suspended in a propellant. Alternatively, the formulation can be a wet aerosol, i.e., a liquid aerosol of the type produced from an aqueous solution of insulin by a liquid nebulae system (see Laube, Journal of Aerosol Medicine, Volt 4, No. 3, 1991, and U.S. Pat. No. 5,320,094, herein incorporated by reference in their entirety). The exact aerosol formulation is not believed to be particularly critical, such that the powder can be in the form of a dry powder or a wet insulin-containing aerosol of the type produced by a nebulizer, so long as the particle size, whether liquid or dry, is of a size which facilitates penetration to the deep lung, in which it is believed that the alveoli serve as the portals from the lungs to the blood. Generally, such a particle size is less than about 10 μm. “Inhaled insulin” is to be contrasted with “intranasally administered insulin” in which insulin is administered within the nasal passages and is weakly absorbed into the bloodstream through the nasal mucosa.

Inhaled insulin, in this invention, is employed to reduce hepatic glucose output, i.e., to induce blood glucose concentrations to fluctuate within a relatively narrow range of 70 to 140 mg glucose/dL and/or HbA1 c to be less than 7%, especially in fasted diabetic patients who have not eaten for at least 6 hours. In such patients, the condition of being outside euglycemia occurs most frequently and/or the magnitude of the departure from normalcy is greater when such patients are in a fasted condition, having not imbibed nourishment (eaten or drank) for at least six hours.

It is preferred to administer inhaled insulin from the time that a patient is either diagnosed to be at risk for being outside euglycemia, or actually diagnosed to actually be outside euglycemia. Inhaled insulin treatment should be maintained until glucose levels have returned to acceptable levels as determined by the attending physician or, failing such a return to levels deemed satisfactory by the physician, permanently.

### DETAILED DESCRIPTION

A. Inhalers/Administration

Any inhaler known to the art may be used for this invention so long as it is capable of delivering a therapeutically effective dose of insulin to the deep lung. This includes any device such as those, which are classified as dry powder inhalers, nebulizers, and metered dose inhalers. Potentially useful are art-recognized inhalers such as those sold under the names Turbohaler (Astra), Rotahaler® (Glaxo), Diskus® (Glaxo), the Ultravent nebulizer (Mallinckrodt), the Acorn II nebulizer (Marquest medical Products), the Ventolin® metered dose inhaler (Glaxo), the Spinhaler® powder inhaler (Fisons), or the like.

In a preferred embodiment, insulin is inhaled as a dry powder by means of a hand-held device such as that disclosed in any of U.S. Pat. Nos. 6,089,228, 5,458,135, 5,775,320, 5,785,049, 5,740,794, and WO 93/00951, the full disclosures of which are herein incorporated by reference. Such devices are available from Inhale Therapeutics Systems, San Carlos, Calif.

B. Formulations

Any formulation, which makes it possible to produce aerosolized forms of insulin, which can be inhaled and delivered to a patient via the intrapulmonary route, can be used in connection with the present invention. Specific information regarding formulations, which can be used in connection with, aerosolized delivery devices are described within Remington's Pharmaceutical Sciences, A. R. Gennaro editor (latest edition) Mack Publishing Company. Regarding insulin formulations, it is also useful to note Sciarra et al. [Journal of Pharmaceutical Sciences, Vol. 65, No. 4, 1976].

A variety of different insulin-containing aerosol formulations can be used in connection with the present invention. The active ingredient within such formulations is insulin which is preferably recombinantly produced human insulin, but which may include insulin extracted from animal sources. Further, the insulin may be an insulin analog, which is an analog of human insulin, which has been recombinantly produced. Although the insulin and/or analog may be present by itself as the sole active ingredient, the insulin may be present with an additional active ingredient such as a sulfonylurea. However, such active ingredients are generally administered separately in order to more closely control dosing and serum glucose levels.

Regardless of the active ingredient, there are several basic types of insulin formulations, which can be used in connection with the present invention. All of the formulations include insulin, preferably with a pharmaceutically acceptable carrier suitable for intrapulmonary administration. In accordance with a first formulation, a low boiling point, highly volatile propellant is combined with the active ingredient and a pharmaceutically acceptable excipient. The active ingredient may, for example, be provided as a suspension or dry powder in the propellant, or the active ingredient may be dissolved in solution within the propellant. Both of these formulations may be readily included within a container, which has a valve as its only opening. Since the propellant is highly volatile, i.e., has a low boiling point, the contents of the container will be under pressure. Thus, when low boiling point propellants are used, the propellants are held within a pressurized canister of the device and maintained in a liquid state. When the valve is actuated, the propellant is released and forces the active ingredient from the canister along with the propellant. The propellant will “flash” upon exposure to the surrounding atmosphere, i.e., the propellant immediately evaporates. The flashing occurs so rapidly that it is essentially pure active ingredient, which is actually delivered to the lungs of the patient. The “flashing” phenomenon which occurs with the use of low boiling point propellants may greatly increase the practicality of the present invention as compared with nebulizers or formulations which do not use such propellants in that larger amounts of drug can be easily administered in a short period of time. Further, since the material being delivered to the lungs is essentially pure drug, it is easier to monitor and more closely control dosing which is a critical feature of the methodology of the present invention. Accordingly, when using such a delivery device it is preferable to use low boiling point propellants such as low boiling point chlorofluorocarbons or hydrocarbons, e.g., trichlorofluoromethane and dichlorodifluoromethane. As non-chlorofluorocarbon containing propellants are developed which are low boiling point propellants, their use in connection with the present invention will become apparent to those skilled in the art.

In accordance with a second embodiment, the insulin is provided in a solution formulation. In this embodiment a dry powder is preferably dissolved in an aqueous solvent to create a solution, which is moved through a porous membrane to create an aerosol for inhalation. Such solutions can be of the type, which are made available commercially for injection and/or other solutions, which are more acceptable for intrapulmonary delivery. An example of a suitable solution for generating aqueous aerosols from a nebulizer is the 0.9% saline solution disclosed in Laube, U.S. Pat. No. 5,320,094.

A preferred form for inhaled administration of insulin is as a dry powder. Preferred insulin dry powders include those, which are described in U.S. Pat. No. 5,997,848 to Patton et al. Such insulin powders comprise free flowing particulates having a size selected to permit penetration into the alveoli of the lungs, generally being less than 10 μm in diameter, preferably less than 7.5 μm, most preferably less than 5 μm, and usually being in the range from 0.1 μm to 5 μm in diameter. Preferably, the insulin particle size is in the range of 0.5 to 3.5 μm. The aforementioned particle sizes generally apply to solid particles. It is also feasible to employ larger size particles which are aerodynamically light but which have a mean diameter much larger than 10 um, say 5 to 30 um. Such particles generally have a low tap density, less than 0.4 g/cm, and a mean diameter between 1 and three microns. Such particles are disclosed in U.S. Pat. RE37,053 E, and in PCT published application WO 01/13891, both herein incorporated by reference. In any case, the insulin powder employed should be of a size that is adapted to penetrating to the deep lung where it can be absorbed through the alveoli.

Alternatively, amorphous insulins could be prepared by lyophilization (freeze-drying), vacuum drying, or evaporative drying of a suitable insulin solution under conditions to produce the amorphous structure. The amorphous insulin so produced can then be ground or milled to produce particles within the desired size range. Crystalline dry powder insulins may be formed by grinding or jet milling of bulk crystalline insulin. The preferred method for forming insulin powders comprising particulates in the desired size range is spray drying, where pure, bulk insulin (usually in a crystalline form) is first dissolved in a physiologically acceptable aqueous buffer, typically a citrate buffer having a pH in the range from about 2 to 9. The insulin is dissolved at a concentration from 0.01% by weight to 1% by weight, usually from 0.1% to 0.2%. The solutions may then be spray dried in conventional spray drying equipment from commercial suppliers, such as Buchi, Niro, and the like, resulting in a substantially amorphous particulate product.

The dry insulin powders may consist essentially of insulin particles within the requisite size range and be substantially free from any other biologically active components, pharmaceutical carriers, and the like. Such “neat” formulations may include minor components, such as preservatives, present in low amounts, typically below 10% by weight and usually below 5% by weight. Using such neat formulations, the number of inhalations required for even high dosages can be substantially reduced, often to only a single breath.

The insulin powders useful in the present invention may optionally be combined with pharmaceutical carriers or excipients, which are suitable for respiratory and pulmonary administration. Such carriers may serve simply as bulking agents when it is desired to reduce the insulin concentration in the powder which is being delivered to a patient, but may also serve to enhance the stability of the insulin compositions and to improve the dispersibility of the powder within a powder dispersion device in order to provide more efficient and reproducible delivery of the insulin and to improve handling characteristics of the insulin such as flowability and consistency to facilitate manufacturing and powder filling.

Suitable carrier materials may be in the form of an amorphous powder, a crystalline powder, or a combination of amorphous and crystalline powders. Suitable materials include carbohydrates, e.g. monosaccharides such as fructose, galactose, glucose, D-mannose, sorbose, and the like; disaccharides, such as lactose, trehalose, cellobiose, and the like; cyclodextrins, such as 2-hydroxypropyl-β-cyclodextrin; and polysaccharides, such as raffinose, maltodextrins, dextrans, and the like; (b) amino acids, such as glycine, arginine, aspartic acid, glutamic acid, cysteine, lysine, and the like; (c) organic salts prepared from organic acids and bases, such as sodium citrate, sodium ascorbate, magnesium gluconate, sodium gluconate, tromethamine hydrochloride, and the like; (d) peptides and proteins, such as aspartame, human serum albumin, gelatin, and the like; (e) alditols, such as mannitol, xylitol, and the like. A preferred group of carriers includes lactose, trehalose, raffinose, maltodextrins, glycine, sodium citrate, tromethamine hydrochloride, human serum albumin, and mannitol.

Such carrier materials may be combined with the insulin prior to spray drying, i.e., by adding the carrier material to the buffer solution which is prepared for spray drying. In that way, the carrier material will be formed simultaneously with and as part of the insulin particles. Typically, when the carrier is formed by spray drying together with the insulin, the insulin will be present in each individual particle at a weight percent in the range from 5% to 95%, preferably from 20% to 80%. The remainder of the particle will primarily be carrier material (typically being from 5% to 95%, usually being from 20% to 80% by weight), but will also include buffer(s) and may include other components as described above. The presence of carrier material in the particles which are delivered to the alveolar region of the lung (i.e., those in the requisite size range below 10 μm) has been found not to significantly interfere with systemic absorption of insulin.

Alternatively, the carriers may be separately prepared in a dry powder form and combined with the dry powder insulin by blending. The separately prepared powder carriers will usually be crystalline (to avoid water absorption), but might in some cases be amorphous or mixtures of crystalline and amorphous forms. The size of the carrier particles may be selected to improve the flowability of the insulin powder, typically being in the range from 25 μm to 100 μm. Carrier particles in this size range will generally not penetrate into the alveolar region of the lung and will often separate from the insulin in the delivery device prior to inhalation. Thus, the particles, which penetrate into the alveolar region of the lung, will consist essentially of insulin and buffer. A preferred carrier material is crystalline mannitol having a size in the above-stated range.

The dry insulin powders useful in the present inventions may also be combined with other active components. For example, it may be desirable to combine small amounts of amylin or active amylin analogues in the insulin powders to improve the treatment of diabetes. Amylin is a hormone, which is secreted with insulin from the pancreatic βcells in normal (non-diabetic) individuals. It is believed that amylin modulates insulin activity in vivo, and it has been proposed that simultaneous administration of amylin with insulin could improve blood glucose control. Combining dry powder amylin with insulin in the compositions of the present invention will provide a particularly convenient product for achieving such simultaneous administration. Amylin may be combined with insulin at from 0.1% by weight to 10% by weight (based on the total weight of insulin in a dose), preferably from 0.5% by weight to 2.5% by weight. Amylin is available from commercial suppliers, such as Amylin Corporation, San Diego, Calif., and can be readily formulated in the compositions of the present invention. For example, amylin may be dissolved in aqueous or other suitable solutions together with the insulin, and optionally carriers, and the solution spray dried to produce the powder product.

The dry powder insulin compositions useful in the present invention are preferably aerosolized by dispersion in a flowing air or other physiologically acceptable gas stream in a conventional manner. One system suitable for such dispersion is described in U.S. Pat. Nos. 5,458,135 and 5,775,320, the full disclosures of which are incorporated herein by reference. The full operation of such a device is disclosed therein.

A preferred dry powder formulation, particularly for use with the aforementioned inhaler, and which was used in the study described below, is disclosed in WO 98/16205 (the full text of which is herein incorporated by reference) as example 3. It consists of a dry powder made by spray drying a formulation containing 7.50 mg human insulin, 1.27 mg mannitol, 3.38 mg sodium citrate, 0.026 mg sodium hydroxide, and 0.32 mg glycine per milliliter of deionized water, for a total solids concentration of 12.5 mg/mL at pH 7.3, the formulation being spray dried to produce a dry powder having an average particle size less than 5 μm. The powder is delivered to the deep lung via an inhaler. For clinical use, this formulation is delivered in two different strengths: a “3 mg” blister containing 3 mg human insulin, 0.50 mg mannitol, 1.35 mg sodium citrate, 0.017 mg sodium hydroxide, and 0.130 mg glycine for a total of 5 mg of anhydrous powder, or a “1 mg” blister containing 1 mg human insulin, 0.167 mg mannitol, 0.45 mg sodium citrate, 0.006 mg sodium hydroxide, and 0.043 mg of glycine for a total of 1.67 mg of anhydrous powder per blister.

The preferred mode of treatment with dry powder insulin is as described in the aforementioned U.S. Pat. No. 5,997,848.

C. Tests

The tests used herein to assess the state of a patient, and whether or not a regimen of inhaled insulin is appropriate, are the well known, art-recognized measurement of fasting and/or random serum, plasma, or whole blood glucose measurements. Using such methods, glucose concentrations and plasma or serum insulin concentrations (the latter usually measured by radioimmunoassay or related techniques) are sampled before and after the administration of a known amount of glucose orally or intravenously. There are known normal patterns of glucose/insulin response to glucose challenge. Normal fasting glucose levels normally fall within the range of 80-126 milligrams per deciliter, mg/dl. Glucose levels in excess of 126 mg/dl may be sufficient for initiating a patient on inhaled insulin or switching a patient from a therapeutic regimen of subcutaneous insulin to a regimen involving inhaled insulin. Laboratory methods for measuring glucose and insulin levels are widely commercially available.

The amount of insulin to be administered via inhalation and the appropriate regimen will generally be determined by the attending physician. In general, when administering insulin in the form of an aerosol, as recombinantly produced human insulin plus excipients, a patient will be administered an amount of inhaled insulin in the range of 0.5 to 50 mg per day, usually 0.5 to 25 mg per day, usually in 1 to 4 individual dry powder doses. The dosing event itself will usually include administering the required dose in one or more, usually 1 to 4, inhalations from a suitable inhaler or nebulizer. Regardless of the form in which the inhaled insulin is delivered, i.e., regardless of whether the insulin is delivered as a dry powder, as an aqueous aerosol produced by a nebulizer, or as a suspension in a propellant delivered from a metered dose inhaler, a patient will be delivered an amount of insulin equivalent to between 1.5 and 150 units delivered systemically (1 mg of inhaled insulin being generally equivalent to about 3 Units (U) of systemically delivered recombinant human insulin). Insulin analogs, which are superactive, can be administered in substantially smaller amounts while obtaining substantially the same effect with respect to reducing serum glucose levels.

The achievement of euglycemia was shown in a clinical study in which subjects with type 2 diabetes mellitus were tested to: 1. To determine whether glycemic control can be achieved at least as effectively with an inhaled insulin regimen as with a conventional subcutaneous (SC) insulin regimen. 2. To assess the toleration and safety of inhaled insulin, and its effects on measures of pulmonary function after 6 months' exposure.

The study was an open-label, randomized, 6-month (24-week), parallel outpatient study with a 4-week lead-in period in subjects with type 2 diabetes mellitus. After screening, subjects maintained a control SC insulin regimen consisting of twice daily (BID) administration of mixed regular insulin and NPH insulin. After the lead-in period, subjects were randomized to receive either the SC injected insulin regimen or an inhaled insulin regimen (pre-meal inhaled insulin (TID) plus a single bedtime Ultralente® injection) for the next 24 weeks.

The subjects evaluated are summarized according to the following table:

| Evaluation Groups: | Inhaled Insulin | SC Insulin |
|---|---|---|
| Randomized | 149 | 150 |
| Treated | 149 | 149 |
| Completed | 132 | 140 |
| Discontinued | 17 | 9 |
| Evaluated for Efficacy: | 146 | 149 |
| Per Protocol Set (Evaluable) | 143 | 145 |
| Assessed for Safety: Adverse Events | 149 | 149 |
| Laboratory Tests | 135 | 142 |
| Pulmonary Function Tests | 145 | 145 |

The criteria for inclusion in the trial were as follows. Male and female subjects aged 35-80 years inclusive with type 2 (not insulin-dependent) diabetes mellitus for >1 year, currently on a stable subcutaneous (SC) insulin administration schedule (at least BID for the 2 months prior to screening). Subjects were required to have screening and pre-randomization glycosylated hemoglobin (HbA1c) between 6% and 11%, inclusive.

Insulin was administered according to the following schedule

Dosage Form Insulin 1 mg aerosolized powder or

Insulin 3 mg aerosolized powder

Ultralente® subcutaneous injection (Eli Lilly Co.)

Inhaler Device Type P3 (Inhale Therapeutics)

Dosing 4-week lead-in period consisting of control SC insulin regimen of twice daily (BID) administration of mixed regular insulin and NPH (?) insulin. After randomization, either pre-meal (TID) inhaled insulin and a single bedtime SC injection of Ultralente®, or the control SC insulin regimen.

Duration 24 weeks (6 months)

Efficacy and safety evaluations were conducted as follows. The primary efficacy endpoint was the change in HbA1 c from baseline to week 24 of treatment. HbA1 c was collected at weeks −4, −1, 0, 6, 12, and 24. Secondary efficacy endpoints included percentage of subjects achieving acceptable glycemic control (HbA1c<8% or <7% at week 24), change in fasting plasma glucose, 2-hour post-prandial glucose and insulin increments following a standardized meal (baseline, week 24), body weight (week −4 and every 4 weeks thereafter), fasting lipids (weeks 0 and 24), and home glucose monitoring. The incidence and severity of hypoglycemic events were monitored. A survey of treatment satisfaction and preference was also administered (weeks −4, −1, 6, 12, and 24).

Safety evaluations included: a full physical examination at screening and brief physical examination (including throat, chest, blood pressure, and heart rate) at week 0 and weeks 4, 8, 16, and 24; 12-lead ECG (screening and week 24); chest X-ray (screening, week 24); clinical laboratory safety tests (screening and week 24); insulin antibodies (weeks 0 and 24); and a pregnancy test for women of childbearing potential (screening). Comprehensive pulmonary function testing (spirometry, lung volumes, diffusion capacity, and oxygen saturation) was performed at baseline (week −3) and at week 24 (spirometry also at week 12). Observed and volunteered adverse events were recorded. At selected sites, a high resolution computerized tomography scan of the thorax was performed on a subset of subjects at baseline and week 24.

The statistical methods employed are described as follows. The primary efficacy endpoint was the week 24 change from baseline in HbA1 c, which was analyzed for both the Full Analysis set (ITT), and the Per Protocol set (Evaluable: the primary analysis population). An analysis of covariance (ANCOVA) model with terms for baseline HbA1 c, center, and treatment was fitted to the week 24 change from baseline HbA1c values. If the week 24 HbA1c was not available, the last evaluable post-baseline HbA1c value was carried forward (LOCF). Non-inferiority of inhaled insulin to subcutaneous insulin was concluded if the upper limit of the 95% confidence interval was less than 0.5%, the protocol-specified non-inferiority bound.

Treatment effects for continuous secondary efficacy parameters were estimated using an ANCOVA model similar to the primary model for HbA1 c. The percent of subjects reaching pre-defined glycemic control goals (HbA1c<8% and <7%) at week 24 was analyzed using logistic regression. The hypoglycemic event risk ratio was estimated using the survival analysis counting process approach for recurrent events, where the analysis model included only a term for treatment.

Efficacy Results: Except where indicated, results are shown for the Evaluable analysis set.

Mean Baseline (BL), Value at Week 24 (LOCF), and Mean Change from BL at Week 24 (LOCF)

| | Inhaled Insulin (INH) | | | SC Insulin | | | Adj. INH-SC | |
|---|---|---|---|---|---|---|---|---|
| | BL | Wk24 | Change | BL | Wk 12 | Change | Difference^{a} | 95% Cl^{a} |
| Primary Efficacy | | | | | | | | |
| HbA1c (%) | 8.1 | 7.4 | −0.7 | 8.2 | 7.6 | −0.6 | −0.07 | −0.32, 0.17 |
| HbA1c (%): (ITT set) | 8.1 | 7.4 | −0.7 | 8.2 | 7.6 | −0.6 | −0.07 | −0.31, 0.17 |
| Secondary Efficacy | | | | | | | | |
| Fasting Glucose | 152 | 132 | −20 | 158 | 149 | −9 | −15.88 | −26.61, −5.15 |
| Glucose Increment^{b} | 89 | 90 | 1 | 94 | 83 | −11 | 6.58 | −8.79, 21.94 |
| Body Weight (kg) | 90.5 | 90.5 | 0.1 | 89.2 | 90.6 | 1.4 | −1.29 | −1.98, −0.59 |

| Hypoglycemia | # Events | Rate | # Events | Rate | INH/SC Risk Ratio | |
|---|---|---|---|---|---|---|
| Total Events^{c} | 1104 | 1.4 | 1278 | 1.6 | 0.89 | 0.82, 0.97 |
| Severe Events^{d} | 4 | 0.5 | 1 | 0.1 | 4.07 | 0.46, 36.43 |

^{a}Difference in adjusted mean change from baseline, and 95% Cl based on primary model.
^{b}Change in concentration (mg/dL) between −30 min and 2 hours post-prandial, measured in standardized meal study.
^{c}Crude event rate was calculated as number of events/total subject-months exposure.
^{d}Crude event rate was calculated as number of events/100 subject-months exposure.

Baseline and End-of-Study HbA1c; Number (%) of Subjects

| | HbA1c: | | | |
|---|---|---|---|---|
| | <7% | 7% to <8% | 8% to <9% | >9% |
| Inhaled insulin | | | | |
| (n = 143) | | | | |
| Baseline | 25 (17.5) | 45 (31.5) | 47 (32.9) | 26 (18.2) |
| End of Study | 67 (46.9) | 42 (29.4) | 15 (10.5) | 19 (13.3) |
| SC insulin (n = 145) | | | | |
| Baseline | 17 (11.7) | 51 (35.2) | 45 (31.0) | 32 (22.1) |
| End of Study | 46 (31.7) | 54 (37.2) | 33 (22.8) | 12 (8.3) |

The average subject-reported daily insulin doses used during the study (inhaled or SC short-acting and SC long-acting) are summarized below.

| | Mean Daily Insulin Use Over Time | | | |
|---|---|---|---|---|
| | Inhaled Insulin N = 157 | | SC Insulin N = 155 | |
| | Short-Acting | Long-Acting | Short-Acting | Long-Acting |
| Baseline* | 23.2 U | 46.5 U | 21.8 U | 45.3 U |
| Week 6 | 15.0 mg | 34.0 U | 24.0 U | 50.1 U |
| Week 12 | 15.5 mg | 36.2 U | 24.7 U | 51.6 U |
| Week 24 | 16.6 mg | 37.9 U | 25.5 U | 52.3 U |

*Baseline dosing was with SC insulin in both treatment groups

The use of both short- and long-acting insulins at baseline were similar between the two treatment groups at baseline. In the inhaled insulin group, the mean daily use of short-acting (inhaled) insulin changed only slightly (15.0 mg to 16.6 mg) during the study. The protocol-defined administration of only 1 injection of long-acting Ultralente® daily resulted in the inhaled insulin subjects using lower doses of long-acting insulin during the study compared to baseline. In the SC insulin group, the average total daily insulin use reported during the study at weeks 6, 12, and 24 generally remained constant.

The average dosing in both treatment groups was relatively constant for these dosing periods over the duration of the study. More subjects in the inhaled group reported using short-acting insulin for pre-lunch, bedtime, and snacks than in the SC group.

Insulin administration frequency was comparable between the groups at baseline (approximately 94% 1-2 times per day and approximately 6% 3-4 times per day). At study weeks 6, 12, and 24, approximately 98% of inhaled insulin subjects were administering insulin by the protocol-specified 3-4 times daily (3 doses of inhaled insulin and 1 bedtime dose of Ultralente®), with the remaining subjects using the inhaled insulin 1-2 times or 5-6 times per day. The SC insulin subjects continued administering insulin at a frequency similar to baseline.

Based on the efficacy, safety, and subject satisfaction results, this study supports the conclusion that inhaled insulin will be a valuable therapy for patients with type 2 diabetes mellitus. Fasting glycemia was significantly lower at study end in patients on inhaled insulin compared to the s.c. group despite its short duration of faction, concomitant doses of long-acting insulin which were lower than with s.c. short-acting insulin, and without more frequent hypoglycemia. Also, the number of patients with optimal glucose control (HbA1 c less than 7%) was higher in the group that used inhaled insulin. It is believed that reduction and/or elimination of hepatic glucose production in fasted patients is, at least in part, responsible for these observations.

What is claimed is:

1. A method of reducing hepatic glucose production in a diabetic patient in need of such treatment who is using exogenous insulin to control blood sugars and who is taking said insulin by the subcutaneous and/or transdermal route of administration, comprising administering said insulin to said patient by the pulmonary route.

2. A method as defined in claim 1, wherein said pulmonary insulin is delivered in the form of aerosolized insulin wherein the aerosol is atomized from a solution.

3. A method as defined in claim 1, wherein said pulmonary insulin is administered as a dry powder.

4. A method as defined in claim 1, wherein said patient has been using exogenous insulin by a subcutaneous route.

5. A method as defined in claim 1, wherein said patient has been using exogenous insulin by a transdermal route.

6. A method as defined in claim 1, wherein said effective amount of pulmonary insulin is delivered by a propellant as a solution or suspension of said insulin from a metered dose inhaler.
